(19) United States
(12) Patent Application Publication
(10) Pub. No.: US 2003/0028231 A1
(43) Pub. Date: Feb. 6, 2003

(54) Radiopaque drug collar for implantable endocardial leads

(75) Inventors: Scott M. Partridge, Blaine, MN (US); Jon Schell, Brooklyn Park, MN (US)

Correspondence Address: NIKOLAI & MERSEREAU, P.A., 900 SECOND AVENUE SOUTH, SUITE 820, MINNEAPOLIS, MN, 55402, US

(73) Assignee: Cardiac Pacemakers, Inc., St. Paul, MN (US)

(21) Appl. No.: 09/920,197

(22) Filed: Aug. 1, 2001

(51) Int. Cl. A61N 1/05

(52) U.S. Cl. 607/120

(57) ABSTRACT

An apparatus for elution of drugs and radiographic visualization that is formed from a polymeric material mixed with a drug and a radiopaque material. The drug and radiopaque material are dispersed through the polymer and the combination is formed into a solid shape having structural integrity.

### BACKGROUND OF THE INVENTION

1. Field of the Invention

The present invention relates generally to implantable leads and, more particularly, to drug elution from and visualization of implanted medical leads.

2. Description of the Related Art

Electrical stimulation using various electrical leads implanted within a patient's body is widely utilized for a variety of indications. For example, pacing leads are frequently used to confer electrical stimuli to the heart and sense electrical stimuli generated by the heart. Cardiac leads are typically introduced transvenously and are fixed at a target location using either active or passive fixation.

To monitor the lead within the patient, radiopaque marker bands or other radiopaque components in the lead may be visualized using a fluoroscope or using other visualization techniques. The visible radiopaque bands or other radiopaque components allow the physician to guide the leads to the heart and to position the distal tip of the lead within the heart.

Once implanted, interactions between the lead and body can vitiate the stimulation's desired effects. For example, material reactions may encourage fibrosis. In regards to pacing, fibrosis is considered a factor in the increase in chronic stimulation threshold that may be experienced over time. Also, the mechanical trauma of implantation can result in inflammation of the adjacent tissue. This inflammation can further alter the response of the tissue to the pacing stimulus, both acutely and chronically.

Other interactions between the lead and body, while not directly affecting the tissue's response to stimulation, are nonetheless undesirable. In some circumstances the body portion to be stimulated may be irritable. The implantation of a lead can compound this irritability. For example, the presence of an implanted lead can promote thrombus formation.

Therefore, in addition to the marker bands, a drug impregnated polymer element may be positioned at or adjacent to the lead's distal end. The drug-impregnated element releases the selected drug, usually a steroid, adjacent to the point of fixation. The drug or a combination of drugs from the collar is used to avoid acute and chronic increases in the stimulation threshold caused by inflammation and/or fibrosis. In addition, thrombus formation may generally be avoided or reduced by the administration of suitable drugs. Regardless of the drugs purpose, a threshold dose of the drug must be provided in order to evoke the desired effect. To reach the threshold, a particular volume of drug-impregnated polymer must be provided at the lead's distal end. Although a lead having both marker bands and drug elution capabilities may be desirable, the distal end of the lead is frequently component dense. As the size of leads is reduced, the space available for drug-impregnated polymer will be even further reduced. The presence of a marker band at the distal end of the lead reduces the amount of drug impregnated polymer that may secured at an appropriate position at the lead's distal end. Therefore, a need exists for a composite material that provides a radiopaque marker and elutes a desired drug. This need is particularly pronounced in leads otherwise having a limited number of radiopaque components.

Further, the need for the drug elution collar to be positioned directly adjacent to the target tissue may require that the marker band be positioned proximally to the collar. Having the marker band proximal to the distal end of a lead can inhibit the ability of a physician to precisely position the distal end because of the end's invisibility. Therefore, a need exists for providing an apparatus that carries out the function of both drug elution and marking.

### SUMMARY OF THE INVENTION

The present invention satisfies the above-mentioned needs and provides additional improvements and advantages that will be recognized by those skilled in the art upon review of the following specification and figures. The present invention provides an apparatus and method for combining radio-opacity with drug elution.

The preferred embodiment comprises an implantable medical lead that includes a lead body having a proximal end and a distal end and at least one elongated electrical conductor contained within the lead body and extending between the proximal end and the distal end thereof. An electrode is disposed at the distal end of the lead body and is connected to the electrical conductor. A combination radiopaque and drug eluting collar is disposed on the lead body immediately proximal of the electrode whereby the distal end of the medical lead can be readily viewed fluoroscopically and a suitable drug is released, over time, from the same area occupied by the radiopaque marker band.

### BRIEF DESCRIPTION OF THE DRAWINGS

FIG. 1 illustrates a pacing lead in accordance with the present invention; and

FIG. 2 is a greatly enlarged, partial sectional view of an embodiment of a lead in accordance with the present invention.

### DETAILED DESCRIPTION OF THE INVENTION

The present invention is applicable to a variety of medical applications utilizing implantable leads. The invention is described generally in the context of a cardiac pacing lead for illustrative purposes only. The appended claims are not intended to be limited to any specific end use, example or embodiment described in this specification. Those skilled in the art will understand that the present invention may be used in a wide variety of medical applications including, but not limited to, neurostimulation, pacing and defibrillation. Further, the numbers are repeated throughout the figures where the individual elements are substantially identical to one another.

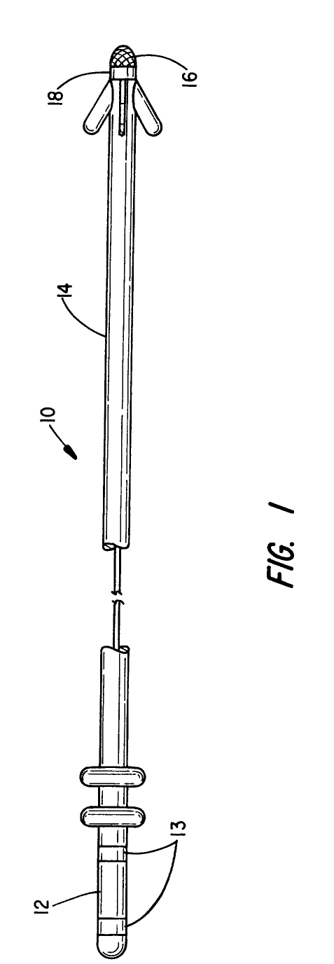

FIG. 1 illustrates a lead in accordance with the present invention. Lead 10 includes at least one proximal connector 12, a lead body 14, at least one electrode 16, and a radiopaque drug-eluting collar 18. In addition, lead 10 includes either an active or a passive fixation mechanism, such as passive fixation tines 20. Connector 12 is located on the proximal end of lead body 14 and is adapted to electrically connect lead 10 to a medical device (not shown), such as a cardiac pacemaker. Connector 12 includes one or more electrical contacts 13 electrically connected to the conductors of lead body 14. The conductors of lead body 14 are electrically connected to distal electrodes 16. Thus, lead body 14 functions to transmit electrical signals between electrodes 16 and the medical device to which lead 10 is attached.

Collar 18 may be a separate element secured to the end of the lead body or may be integrally molded into the distal end of the lead body. Collar 18 may take any number of shapes or configurations that may be attached to or otherwise disposed in the distal end of a lead body. Collar 18 is typically in the shape of a ring that is attached over the exterior surface of lead body 14 or a toroidal insert that is fitted within a cavity at the distal end lead body 14 during manufacture. Collar 18 is generally positioned on lead body 14 to allow a drug eluted from the collar to come into contact with a target tissue proximate electrode 16 and to aid a physician in positioning the lead using fluoroscopy or other visualization methods during implantation. Thus, collar 18 is typically secured to the distal end of lead body 14.

To facilitate both drug elution and radio opacity, collar 18 is constructed of a novel composition. The composition includes a carrier material, a drug and a radiopaque material. The carrier material is typically a silicone rubber or a polymeric matrix, such as polyurethane. Generally, the carrier material is selected and formulated for an ability to incorporate the desired drug during manufacture and release the drug within the patient after implantation. Radiopaque filler materials for creating the collar 18 may include, but are not limited to, barium sulfate, bismuth tungsten in various quantities and/or combinations. Typically, the drug component is steroid, such as a glucocorticosteriod. The amount of any particular drug incorporated into collar 18 is determined by the effect desired, the drugs potency, the rate at which the drug capacity is released from the carrier material, as well as other factors that will be recognized by those skilled in the art. The radiopaque material is selected for biocompatibility and for its visibility when using a particular method of visualization. Tungsten, bismuth, barium sulfate or a combination of these materials is typically used when the implantation is being monitored using fluoroscopy. Alternatively, the radiopaque material may be an iodine based angiographic contrast material such as ioversol, sold as Optiray and manufactured by Mallincrodt, Inc., Iohexol, manufactured by Nycomed, or Iotrolan, manufactured by Scherling. The radiopaque material may be uniformly distributed throughout the collar or alternatively the radiopaque material may be localized within the collar. Thus, the entire collar or a portion of the collar can be mixed or impregnated with the radiopaque material so long as it is sufficient to provide a good image.

A collar in accordance with the present invention may be made by mixing (or dissolving; or melting). The radiopaque filler material and steroid will typically be mixed with uncured silicone rubber. It may include, but is not necessarily limited to, two part liquid silicone rubbers, gum stock silicone rubbers, or medical adhesives used for creating or bonding silicone rubber components. The opaque filler and steroid is added to the uncured silicone rubber in various quantities and following the mixing, the silicone rubber is cured and formed into the collar component for steroid delivery and opacity. Care should be taken that the method selected does not heat the mixture including the drug beyond a point that would destroy the drug. The collar can be formed by any suitable process, including molding, extruding or other suitable processes recognized by those skilled in the art.

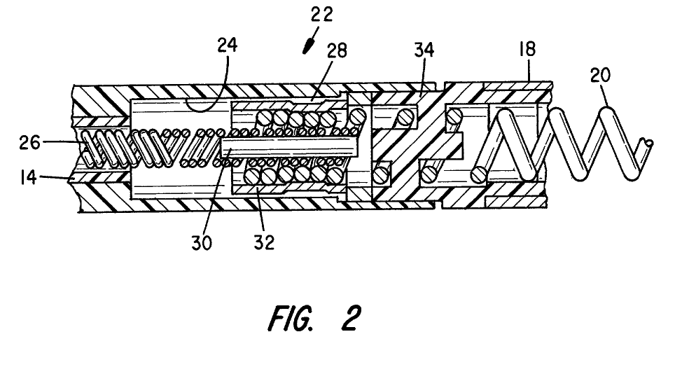

Referring to FIG. 2, there is illustrated in greatly enlarged form, a distal end portion of a medical lead of the positive or active fixation type and incorporating the radiopaque, drug-eluting collar of the present invention. Shown in FIG. 2 is the electrode assembly which is disposed at the distal end portion of a medical lead and which incorporates a tissue piercing helix 20 as an electrode. The head portion 22 of the lead body 14 includes an internal lumen 24 and entering the lumen from its proximal end is an elongated, multi-filar coil 26 shown as having three individual filars. Surrounding the coil 26 is a tubular sheath 14 that comprises the lead body and which extends to the proximal end of the lead. The coil 26 is free to rotate within the lead body 14 and its proximal end portion electrically connects to the proximal end portion 28 of the helix 20. More particularly, a solid tubular stem 30 is disposed in a lumen formed by the turns of the multi-filar coil 26 and a crimp tube 32 is placed about the proximal end portion 28 of the helix 20 and when compressed, mechanically and electrically joins the proximal turns of the helix 20 to the turns of the multi-filar coil.

Also contained within the lumen 24 of the head portion 22 of the lead is an electrode guide 34 that is formed of a molded plastic.

As the coil conductor 26 within the lead body 14 is rotated, in a counterclockwise direction when viewed from the distal end of the lead, the helix electrode 20 is effectively screwed out of the distal end of the lead as it rotates around the electrode guide 34. In this fashion, the electrode can be screwed into tissue, much like a corkscrew, to more positively secure the lead to the tissue.

In accordance with the present invention, there is also provided at the distal end of the lead's head member 22, the collar 18 which, in accordance with the present invention, comprises both a drug eluting and a radiopaque member. The collar 18 is preferably made in accordance with the methodology described above in connection with the embodiment of FIG. 1 and need not be repeated here.

In use, a collar in accordance with the present invention can be secured to the distal tip of a pacing lead. The pacing lead is inserted transvenously into the patient through the subclavian vein. The physician typically monitors the advancing tip using a fluoroscopic monitoring device. The lead is guided through a series of veins to the heart. By watching the image on monitor, the physician can determine the precise location of the band within the patient because of the radiopaque material incorporated into the collar. Once in the heart, the physician may visualize the precise location of the lead's distal tip. When the tip is positioned at the desired location adjacent the heart wall, the lead is fixed using either an active or a passive fixation mechanism. The drug component of the collar is then dispensed through dissolution directly adjacent to the fixation site to facilitate the drug's intended effect.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

What is claimed is:

1. An implantable medical lead comprising:
(a) a lead body having a proximal end and a distal end and at least one elongated electrical conductor contained within the lead body and extending between the proximal end and the distal end;
(b) an electrode disposed at the distal end and connected to the at least one electrical conductor; and
(c) a radiopaque, drug eluting collar disposed on the lead body immediately proximal of the electrode.

2. The implantable medical lead of claim 1 wherein the radiopaque, drug eluting collar comprises a polymeric carrier incorporating particles of radiopaque material and a drug, the carrier allowing release of the drug over a predetermined time period.

3. The implantable medical lead of claim 2 wherein the radiopaque material is selected from a group consisting of barium sulfate, bismuth tungsten, and mixtures thereof.

4. The implantable medical lead as in claim 2 wherein the drug is selected from a group consisting of anti-thrombogenic, anti-fibrotic, anti-inflammatory and antiarrhythmic agents.

5. The implantable medical lead as in claim 4 wherein the anti-inflammatory agent is dexamethasone sodium phosphate.

6. The implantable medical lead as in claim 2 wherein the polymeric carrier is a silicone rubber matrix.

7. An implantable medical lead, comprising:
a lead body having a proximal end and a distal end; and
a radiopaque, eluting drug collar secured to the distal end of the lead body.

8. An implantable medical lead, as in claim 7, wherein the radiopaque drug eluting collar comprises a carrier material containing at least one drug and at least one radiopaque material.
